(19) United States
(12) Patent Application Publication
(10) Pub. No.: US 2010/0168686 A1
(43) Pub. Date: Jul. 1, 2010

(54) METHOD AND APPARATUS FOR TREATING THE SKIN

(75) Inventors: De Grimaldi Jean-Philippe, London (GB); Daniel Laurent Sister, London (GB)

Correspondence Address: BREINER & BREINER, L.L.C., P.O. BOX 320160, ALEXANDRIA, VA, 22320-0160, US

(73) Assignee: Danakim Investments Limited, Tortola (VG)

(21) Appl. No.: 12/733,122

(22) PCT Filed: Aug. 15, 2008
(86) PCT No.: PCT/GB2008/002767

§ 371 Date: Feb. 12, 2010

(30) Foreign Application Priority Data
Aug. 16, 2007 (GB) 0716030.2

(51) Int. Cl. A61F 7/08; A61M 35/00

(52) U.S. Cl. 604/290; 604/291

(57) ABSTRACT

A method for treating the skin involves a first stage of softening the skin by projecting steam including treatment material on to it, causing the pores to open and the material to penetrate the outer skin surface. Thereafter the pores are caused to close e.g. by projecting an astringent composition on to the skin. Apparatus to effect this is described. One form includes a sealable chamber into which a quantity of liquid treatment preparation may be placed and heated to vaporise it and project the vapour via a nozzle (10) on to the skin surface, at a distance suitable for the treatment and established by a spacer (17) projecting from the side of a housing (1) containing the chamber and nozzle (10), and means (11) to trigger dispensing of the vapour when actuated.

This invention relates to a method for treating the skin and for apparatus for use in that method.

For millennia people have sought to improve their appearance by treating their skin. In particular, a wide variety of materials has been applied to the skin with a view to improving its appearance. Many of these operate by interaction with the skin itself, for example by transfer of components into the skin to change its appearance.

Applying material to the skin surface, while simple to effect, does not always produce an adequate treatment, particularly since it can take some time for active constituents in the applied material to penetrate into the skin. In order to ensure skin penetration, it has previously been proposed to inject active material into the skin using very short injection needles. This is effective, but requires skillful application and hygienic precautions to be taken to prevent any risk of cross-infection when the apparatus is used on a series of people undergoing the treatment.

It is also known to apply treatment materials using steam. For example, U.S. Pat. No. 4,274,588 discloses apparatus for generating steam, and mentions that a cosmetic or pharmaceutical substance can be applied to the skin by using the apparatus. US-A-2006/0207013 A1 discloses a shower-mounted facial treatment cocoon which may be used to apply treatment agents using steam. U.S. Pat. No. 3,949,743 discloses a medicated vapor production method and apparatus including a hood having an open side against which the face of the user may be located.

GB-A-2280848 discloses a mist generator for skin treatment, and the addition of facial softening lotion to the water from which the mist is generated. JP-A-2002263162 discloses a further facial steamer.

We have now found that the efficacy of such steam treatment may be materially improved by using a two-stage method which involves in a first stage projecting hot steam on to the skin to open the pores, projecting treatment material on to the skin and in a second stage thereafter causing the pores to close, so trapping the projected material.

According therefore generally to the present invention, there is provided a method of treating skin which involves carrying out the steps of softening the skin to open the pores by projecting steam including treatment material on to the skin, and which is characterized by thereafter causing the pores of the skin to close with the retention of the treatment material projected therein.

In the first part of the method, a vaporised liquid preparation is blown on to the skin, which softens the skin, cleans it somewhat, and opens the pores, to enable the liquid preparation to penetrate under the outer layer of the skin, carrying the treatment material with it. Most preferably, the treatment material is formulated in an aqueous medium which is heated to boiling point to produce steam which is projected through a nozzle directed at the skin. By suitably controlling the distance between nozzle and skin, the stream of moist air which reaches the skin does so without discomfort because it has cooled down somewhat and the treatment material is simply projected as droplets on to the skin surface, and absorbed through the pores.

After an appropriate amount of treatment material has been projected on to the skin in this way, in the second stage of treatment, the pores are closed by projecting an appropriately astringent composition on to the skin surface. This causes the pores to close, so trapping the active material and allowing it to exert its beneficial effect on the outer layer of the skin.

More than one treatment material may be projected on to the skin simultaneously and different compositions may be adopted for differing skin conditions and desired effects. In the second stage of the method where the pores are caused to close, in addition to projecting something having a astringent effect on to the skin, other material may be projected, for example antiseptic materials or other longer-acting skin treatment materials or compositions.

The method just described is conveniently practised using apparatus specifically configured for the purpose.

In accordance with a second feature of the invention, there is provided apparatus for beauty treatment comprising means for projecting a warm or hot stream of treatment material carried by a stream of vaporised liquid on to a skin surface, the apparatus including a nozzle through which vapour and treatment agent may pass and, located downstream of the nozzle, a spacer adapted to define a distance between the nozzle and the area of skin to be treated, and means for projecting an astringent composition on to the skin surface.

The spacer may be permanently fixed in place as part of the apparatus, but in order for the apparatus to be compacted for storage, or to enable different distances to be specified for different skin treatments, it is preferably removably mountable in the body of the apparatus. In such a case, it is important to provide means to ensure that the stream of treatment material can be dispensed only when the spacer is in position, for example by mounting the nozzle and means for producing the vapour in a body which also includes a receptacle into which a spacer fits, and located adjacent the receptacle, means for detecting when a spacer is so fitted, so enabling the dispensing of the treatment material.

One simple way of achieving this is to mount the spacer in an elongate socket, one end of which is open to allow the spacer to be inserted into it and the other end being associated with a microswitch or like mechanism to detect when a spacer is inserted.

In a particularly preferred embodiment, the apparatus according to the invention comprises a housing, means for mounting a spacer on the housing, a chamber located in the housing, a nozzle connected to an outlet from the chamber, means for heating a quantity of treatment liquid in the chamber and cause it to be projected from the nozzle in the direction of the end of the spacer remote from the housing, means for charging the chamber with a quantity of aqueous based treatment material, means for sealing the chamber, and actuatable valve means between the chamber and the nozzle.

The chamber preferably has associated with it a sealable filling aperture to enable the treatment liquid to be introduced therein. After such introduction, the treatment material is heated in the chamber sufficiently to enable it to be projected as a moist vapour stream when the actuatable valve means between chamber and nozzle is actuated.

In a preferred embodiment, the sealable filling aperture is designed to co-act with a sealed phial of treatment material in such a way that, as a phial is inserted into the aperture, a seal on the phial is first broken, allowing its contents to flow into the chamber, and thereafter, when the phial is fully inserted, the filling aperture is sealed.

The invention is illustrated by way of example with reference to the accompanying drawings in which:

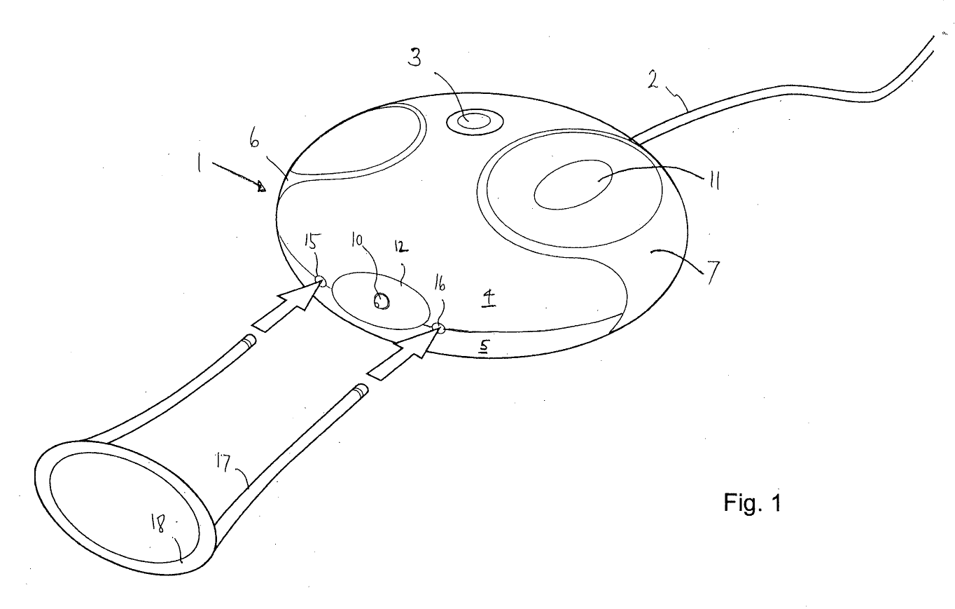

FIG. 1 is a schematic drawing of apparatus according to the present invention prior to use;

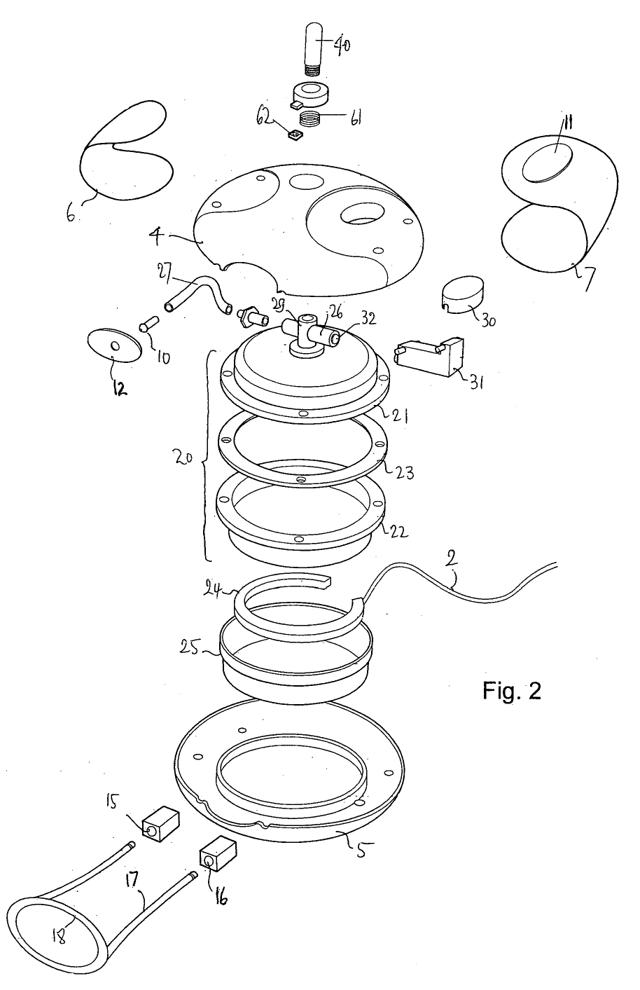

FIG. 2 is a simplified exploded diagram of the apparatus shown in FIG. 1;

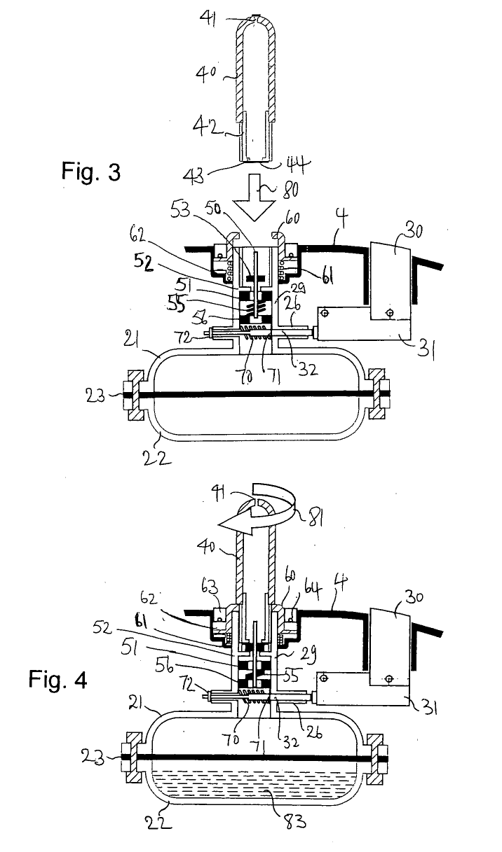

FIG. 3 is a vertical section through part of the apparatus shown in FIG. 1 prior to the insertion of a phial of treatment material;

FIG. 4 is a section similar to FIG. 3 when the apparatus is ready for use; and

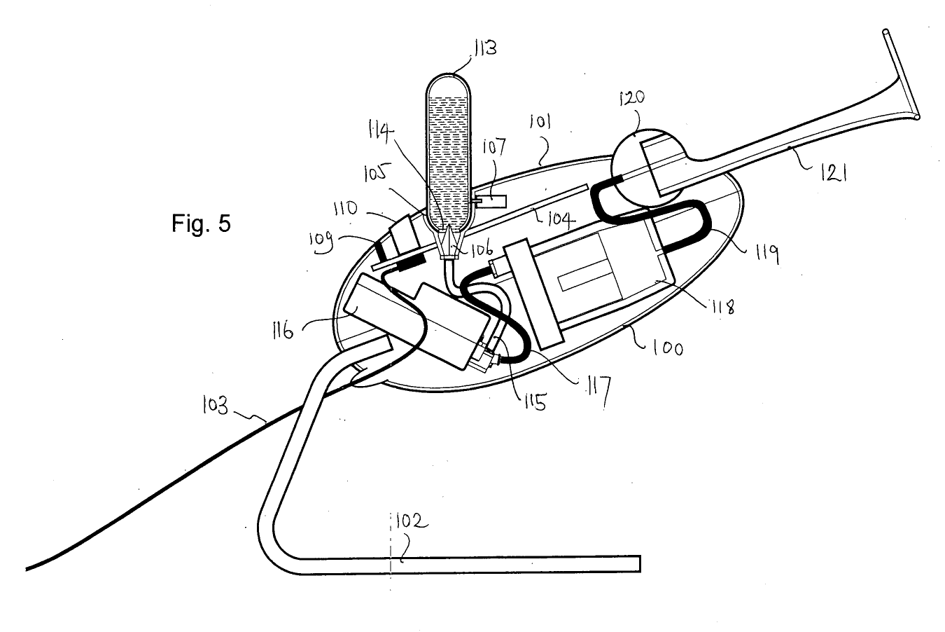

FIG. 5 is a diagrammatic section through an alternative apparatus according to the present invention.

Referring to FIGS. 1 to 4 of the drawings, the apparatus shown consists of a housing 1 containing the various functional components and having extending from its base an electrical supply lead 2 enabling electrical power to be supplied to the components within the housing. The housing consists of top and bottom portions 4, 5 which may be held together by any convenient means.

Located centrally in the top portion 5 is a socket 3 into which a sealed phial of treatment material may be inserted.

Mounted in the side of the housing 1 is a nozzle 10 for projecting hot moist vapour away from the housing itself. Bridging the housing portions 4, 5 are two rubber hand grips 6, 7, one of which incorporates a press pad 11 which is used to trigger release of vapour from nozzle 10. Nozzle 10 is surrounded by a trim plate 12.

To either side of nozzle 10 are two sockets 15, 16 into which the legs of a spacer device 17 may be inserted. Insertion is in the direction of the arrows shown in FIG. 1. The sockets and the legs of the spacer device may be configured in known fashion to enable the legs to be inserted and held, and to be removed when desired.

When so inserted, the spacer device 17, which has an oval or elliptical ring 18 at its end remote from the housing 1, acts to define a treatment distance from the nozzle 10 to the skin when the device is in use. Associated with one or both sockets 15 and 16 are means (not shown) to disable the device when no spacer is inserted.

FIG. 2 shows the general internal arrangements of the device. Located centrally in the housing is a heating chamber 20 formed of upper and lower casings 21, 22, held together by fixings around their periphery and with an interposed a gasket seal 23. The lower casing 22 has a surrounding heating element 24, covered by a plastics heat shield 25. On top of casing 21 is a spigot 29 with a transverse branch 26. One end of branch 26 is connected, via a steam hose 27, with nozzle 10.

Located below press pad 11, when the device is assembled, is a trigger button 30 which sits on a pivotally mounted trigger link 31. The end of link 31 contacts a trigger shaft 32 which extends through branch 26.

The detailed construction of the chamber 20 and its filling and sealing mechanism is shown in FIGS. 3 and 4, together with the construction of the phial of treatment material. This consists of a glass phial 40 having a foil sealed aperture 41 at its upper end. Set into the lower end is an externally threaded aluminium tube 42, with an annular flange 43 at its base covered with a foil seal 44.

The upper end of spigot 29 is internally threaded. Within spigot 29 is a spring mounted piercing needle 50. Needle 50 is fixed to a guide ring 51 which can slide vertically in spigot 29 between an upper position in which it contacts the underside of a flange 52 formed on the internal wall of spigot 29, and a lower position defined by the abutment of a seal ring 53 fixed on needle 50 against the upper side of flange 52. A spring 55 is located between ring 51 and a ring 56 set in spigot 29 just above branch 26 to bias needle 50 to the upper position.

Surrounding the upper end of spigot 29 is a cap ring 60, spring biased upwardly by a compression spring 61 between cap ring 60 and part of housing top portion 4. A microswitch 62 is set on housing portion 4 to enable determination of when cap ring 60 is depressed. Cap ring 60 carries an external translucent plastics collar 63 having two LEDs 64 incorporated in it, one red, one green.

Trigger shaft 32 is biased to the right as shown in FIGS. 3 and 4 by a spring 70 bearing on a clip 71 set on shaft 32. The left-hand end of shaft 32 is of diameter less than the internal diameter of branch 26 and carries a seal ring 72 which seals against the end of branch 26. The right hand end of shaft 32 is a sliding fit in branch 26.

When it is desired to treat the skin, the spacer 17 is first inserted into the sockets 15, 16, the device is connected to an electrical supply using lead 2. A sealed phial of the first treatment material is then inserted into socket 3 (in the direction of arrow 80 in FIG. 3), followed by turning the phial 40 as shown by arrow 81 in FIG. 4 to engage the thread on tube 42 with the internal thread at the top of spigot 29. Immediately after insertion, the foil seal covering aperture 41 is pierced with a pin, or, e.g., scraped off using a fingernail. As the phial is rotated, it is screwed into spigot 29 and needle 50 pierces foil 44, enabling liquid in the phial to pass down through spigot 29 and collect on the base of the chamber, as shown at 83 in FIG. 4. On continued rotation, flange 43 and the remains of seal 44 contact the upper side of seal ring 53 and move seal ring 53 and the needle 50 down until seal ring 53 seals against flange 52.

At the same time, the lower edge of phial 40 contacts the top of cap ring 60 and depresses it, with compression of spring 61. The dimensions are such that as seal ring 53 seals on flange 52, microswitch 62 is actuated, which enables the heater 24, via suitable circuitry, not shown, and lights red LED 64. When the heater has heated up liquid 83 adequately, the red LED 64 extinguishes and the green one lights to show that the device is ready for use.

The contents of the phial may now be projected as steam via nozzle 10 towards ring 18 by depressing press pad 11. This moves trigger shaft 32 to the left (via button 30 and link 31), so moving seal ring 72 away from the end of branch 26 to enable the vaporised content of the phial to pass through branch 26 and steam hose 27 and cause it to be projected as a stream of material through nozzle 10 on to the area of skin within ring 18.

The device includes an appropriate mechanism to ensure that when the contents of the phial has been exhausted, the device ceases to operate.

Once the content of the first phial has been projected on to the skin in this way, the first phial is unscrewed from socket 3 and the process repeated with the second phial to complete the treatment.

The materials in the treatment phials may vary widely depending upon the precise skin treatment to be effected. By way of example, good results may be achieved in skin treatment of the face and neck by using treatment compositions as follows:

### First Phial:

### Composition 1

This composition is for the initial stage of the treatment, i.e. warming and cleansing the skin, opening up the pores, and delivery into those pores of the active ingredients. The following are the % w/w concentrations of the ingredients:

| | |
|---|---|
| Water | 96.5 |
| PPG-26 Buteth-26 | 0.8 |
| PEG-40 Hydrogenated castor oil | 0.7 |
| Lavender Oil | 0.4 |
| Tea Tree Oil | 0.4 |
| Ravensara Oil | 0.4 |
| Helichrysum Oil | 0.2 |
| Camphor Oil | 0.2 |
| Zinc Gluconate | 0.3 |
| Copper Gluconate | 0.1 |

### Second Phial:

### Composition 2

The second treatment composition, to close the pores and provide an antiseptic effect, likewise consists of the following materials in the % w/w proportions indicated:

| | |
|---|---|
| Aqua | 82.5 |
| Sodium Hyaluronate | 8.0 |
| Centella Asiatica Leaf Extract | 2.0 |
| Aloe Barbadensis Leaf Extract | 1.0 |
| Glycerin | 1.0 |
| Hydrolysed Silk | 1.0 |
| Spirulina maxima Extract | 1.0 |
| Lilium Candidum Extract | 1.0 |
| Calendula Officinalis Flower Extract | 0.5 |
| Arnica Montana Flower Extract | 0.5 |
| PPG-26 Buteth-26 | 0.6 |
| PEG-40 Hydrogenated Castor Oil | 0.4 |
| Sodium Ascorbyl Phosphate | 0.1 |
| Methyl Paraben | 0.1 |
| Citric Acid | 0.08 |
| Fragrance | 0.15 |
| Citrus Amara | 0.05 |
| 2-Bromo-2-nitripropane-1,3-diol | 0.02 |
| CI 42090 | 0.0014 |

Referring to FIG. 5, this shows diagrammatically an alternative construction of apparatus in accordance with the present invention.

The apparatus shown consists of a housing formed of an upper body moulding 101 and a lower body moulding 100 fixed together by appropriate means. The lower body moulding 100 includes a slot into which a portion of a detachable stand 102 may be fitted so that the apparatus adopts an upwardly inclined position as shown in the drawing. The lower body moulding 100 also includes an aperture through which a mains electrical lead 103 may pass in order to connect the apparatus to a source of power. Internally of the housing, mains lead 103 is connected to components on a printed circuit board 104 which is located in the upper body moulding.

Not shown in the drawing is an appropriate microswitch mounted in the lower body moulding 100 which is connected to the printed circuit board 104 and which acts to disable the apparatus unless stand 102 is fitted into the lower body moulding as shown.

Located in the upper body moulding 101 is a socket 105 at the base of which is located a pointed seal-piercing member 106. To one side of the socket is located a locking unit 107 which incorporates a microswitch likewise connected to components on the printed circuit board 104. A projecting pin forming part of unit 107 passes through the wall of socket 105 and can hold in place a capsule of treatment liquid. The drawing shows a capsule 113 located in socket 105. Located at the bottom of capsule 113 as shown is a foil seal 114 which is pierced as the capsule is inserted into socket 105.

At the bottom of socket 105, there is an outlet connected via a flexible pipe 115 to the inlet port of an electric pump 116. The outlet port of electric pump 116 is connected via a flexible pipe 117 to an inlet port on a heater block 118. An outlet from the heater block is connected via a flexible pipe 119 to a nozzle 120 which is mounted in the upper body moulding 101. As can be seen in the drawing, nozzle 120 is of spherical shape enabling it to be rotated about its centre through an angular range. Rotation of nozzle 120 is easily effected by moving a detachable guard unit 121 which is fitted into the nozzle 120 and which defines a treatment distance between the nozzle 120 and the face or other skin surface of the user when the apparatus is in use.

Not shown in the drawings is a microswitch arrangement associated with nozzle 120 and connected to the printed circuit board 104 which prevents operation of the apparatus unless guard 121 is in place with its elongate spacing members located in sockets in nozzle 120.

The printed circuit board 104 carries appropriate circuit components to enable the apparatus to function, including a light-emitting diode indicator 109, a light-emitting surface of which is visible, set into the surface of upper body moulding 101, and an actuation switch 110 which is located so that it may be actuated by a user when it is desired to apply steam to the skin.

In use, the apparatus is first assembled as shown in FIG. 5, i.e. stand 102 is inserted into the lower body moulding and guard 121 into the nozzle. The power lead 103 is then connected to a source of electrical power and this causes the heater block 118 to start heating up. An appropriate sensor on block 118 may be connected to the circuitry on the printed circuit board 104 which may be configured to illuminate the LED indicator 109, to show that the apparatus is connected to a source of electrical power, and with indicator 109 glowing red initially and then changing to green when the heater block has reached the desired temperature.

At this point, if not before, a capsule 113 is inserted into socket 105 with rupture of the foil seal 114 enabling treatment liquid to flow out of capsule 113 and through pipe 115 to pump 117. The circuitry is arranged so that actuation switch 110 is inoperative until the heater block is at the right temperature, but once that has been reached, shown by the indicator 109 changing from red to green, the user simply presses actuation switch 110 to start pump 116 working. Pump 116 draws liquid via pipe 115 from the capsule and socket 105 and drives that liquid via pipe 117 to the heater block 118. The heater block has risen to a temperature adequate to vaporise the treatment liquid and, as a result, steam passes out of the heater block through pipe 119 and out of nozzle 120 directed towards the remote end of guard unit 121, where the user has located his or her face.

Pumping continues until the contents of capsule 113 have been exhausted, at which point the pump may switch off automatically and the locking mechanism in unit 107 may then be released to enable the first capsule 113 to be removed. On placing the second treatment capsule into socket 105, the heating and vaporising cycle is repeated, this time to achieve the second stage of the treatment method of the present invention.

1-14. (canceled)

15. A method of treating skin comprising softening skin to open pores of the skin by projecting steam including treatment material onto the skin, and thereafter causing the pores of the skin to close with retention of the treatment material projected therein.

16. The method according to claim 15, wherein the treatment material is projected onto the skin by heating an aqueous medium containing the treatment material to boiling point to produce the steam and projecting the steam through a nozzle directed at the skin.

17. The method according to claim 15, wherein the pores are caused to close by projecting a substantially astringent composition onto the skin surface.

18. The method according to claim 15, wherein more than one treatment material is projected onto the skin simultaneously.

19. The method according to claim 15, wherein as the pores are caused to close, antiseptic material or one or more other skin treatment materials are projected onto the skin.

20. An apparatus for beauty treatment comprising means for projecting a warm or hot stream of treatment material carried by a stream of vaporized liquid onto a skin surface, including a nozzle structured for passage of vapor and treatment agent therethrough, and located downstream of the nozzle, a spacer adapted to define a distance between the nozzle and an area of the skin surface to be treated, and means for projecting an astringent composition onto the skin surface.

21. An apparatus according to claim 20, wherein the spacer is removably mounted in a body of the apparatus.

22. An apparatus according to claim 21 further including means to ensure that the stream of treatment material is projected only when the spacer is in position.

23. An apparatus according to claim 22, wherein the nozzle and means for producing the vaporized liquid are mounted in the body of the apparatus, said body also including a receptacle into which the spacer fits, and located adjacent the receptacle, means for detecting when the spacer is so fitted, and enabling projection of the treatment material only upon such detection.

24. An apparatus according to claim 23, wherein the spacer is mounted in an elongate socket with a first end which is open to allow the spacer to be inserted into the socket and a second end which is associated with a mechanism to detect when the spacer is inserted.

25. An apparatus according to claim 20 further comprising a housing, means for mounting the spacer on the housing, a chamber located in the housing, a nozzle connected to an outlet from the chamber, means for heating a quantity of treatment liquid in the chamber and to cause the treatment liquid to be projected from the nozzle in a direction of an end of the spacer remote from the housing, means for charging the chamber with a quantity of aqueous based treatment material, means for sealing the chamber, and actuatable valve means between the chamber and the nozzle.

26. An apparatus according to claim 25, wherein the chamber has associated therewith a sealable filling aperture to enable the treatment liquid to be introduced therein.

27. An apparatus according to claim 26, wherein the sealable filling aperture is structured to co-act with a sealed vial of said treatment material in such a way that, as the vial is inserted into the aperture, a seal on the vial is broken allowing contents of the vial to flow into the chamber, and thereafter, when the vial is fully inserted, the filling aperture is sealed.

28. An apparatus according to claim 20 further comprising a housing, means for mounting the spacer on the housing, a treatment liquid reservoir adapted to be filled with a quantity of treatment liquid from outside the housing, a heater block, means for pumping the treatment liquid from the reservoir and through the heater block, and nozzle means connected to an outlet on the heater block for directing steam at a location defined by a spacer mounted on the housing.
